(19) United States
(12) Patent Application Publication
(10) Pub. No.: US 2009/0012046 A1
(43) Pub. Date: Jan. 8, 2009

(54) Methods of Treating Mutated Hiv

(76) Inventors: Dirk Edward Desire Jochmans, Herent (BE); Piet Tom Bert Paul Wigerinck, Terhagen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/160,115

(22) PCT Filed: Feb. 5, 2007
(86) PCT No.: PCT/EP07/51087

§ 371 Date: Jul. 7, 2008

(30) Foreign Application Priority Data
Feb. 3, 2006 (EP) 06101297.7
May 30, 2006 (EP) 06114705.4

(51) Int. Cl. C07D 487/04; A61K 31/437; A61K 31/675; A61P 31/18

(52) U.S. Cl. 514/81; 546/86; 514/292

(57) ABSTRACT

Nucleotide-competing reverse transcriptase inhibitors (NcRTI) bind to the active site of HIV reverse transcriptase (RT) in competition with the next incoming nucleotide. To further investigate the impact of RT inhibitor resistance mutations on the activity of NcRTIs, the susceptibility of >6000 recent clinical isolates for a prototype compound, NcRTI-1, was determined. Over 80% of the profiled clinical isolates remained susceptible for NcRTI-1 (FC<4). No cross-resistance was observed between NcRTI-1 and currently used RT inhibitors, apart from limited cross-resistance with 3TC/FTC.

Analysis of the genotype of >1700 of these viruses showed that the combination of active site mutations M184V+Y115F correlated most with resistance to NcRTI-1 (FC=75). Analysis also indicated that the K65R mutation is associated with hypersusceptibility to NcRTI-1 and that it reverses the reduced susceptibility caused by M184V. These findings were confirmed in SDM strains. This reciprocity between the K65R and M184V mutation is unparalleled among RT inhibitors. When replicating wild-type HIV-1 in the presence of NcRTI-1, M184V+Y115F were selected. In the presence of both NcRTI-1 and tenofovir, NcRTI-1 prevents the selection of K65R.

### CROSS REFERENCE TO RELATED APPLICATIONS

This application claims priority of the benefits of the filing of PCT Application No. PCT/EP2007/051087 filed Feb. 5, 2007, European Patent Application No. 06101297.7 filed Feb. 3, 2006 and European Patent Application No. 06114705.4 filed May 30, 2006. The complete disclosures of the aforementioned related patent applications are hereby incorporated herein by reference for all purposes.

### FIELD OF THE INVENTION

This invention relates to methods of treating patients infected with Hunan Immunodeficiency Virus (HIV) that has a K65R mutation in the viral genome encoding for reverse transcriptase.

### BACKGROUND OF THE INVENTION

Drugs that are currently on the market or under development to combat HIV viral infection belong to classes such as reverse transcriptase inhibitors (RTIs), protease inhibitors (PIs) and the more recent fusion inhibitors. RTIs prevent viral replication by intervening in the reverse transcription mechanism while PIs intervene in the viral assembly. RT inhibitors interact with the RT enzyme in a number of ways to inhibit its functioning so that viral replication becomes blocked. PIs bind to the active site of the viral protease enzyme, thereby inhibiting the cleavage of precursor poly proteins necessary to produce the structural and enzymatic components of infectious virons.

Nucleoside Reverse Transcriptase Inhibitors (NRTIs) belong to a class of RT inhibitors that are intracellularly converted to nucleoside triphosphates and compete with the natural nucleoside triphosphates for incorporation into elongating viral DNA by reverse transcriptase. Chemical modifications that distinguish these compounds from natural nucleosides result in DNA chain termination events.

Currently available NRTIs include zidovudine (ZDV or AZT), didanosine (ddl), zalcitabine (ddC), stavudine (d4T), lamivudine (3TC), abacavir (ABC), emtricitabine (FTC). A nucleotide reverse transcriptase inhibitor (N(t)RTI) is tenofovir disoproxil fumarate (TDF). For example, AZT, one of the first HIV RT inhibitors identified, is converted to the triphosphate (TP) by cellular kinases. HIV-1 RT is subsequently able to use AZT-TP as an efficient alternative substrate in the building of the viral DNA.

However, AZT-TP lacks a 3'OH necessary for further DNA elongation, thereby causing termination of the growing DNA chain following incorporation.

Another class of RT inhibitors are the Non-Nucleoside RT Inhibitors (NNRTIs): delavirdine, efavirenz (EFV), and nevirapine (NVP)

Although effective in suppressing HIV, each of these drugs, when used alone, is confronted with the emergence of resistant mutants. This led to the introduction of combination therapy of several anti-HIV agents usually having a different activity profile. In particular the introduction of “HAART” (Highly Active Anti-Retroviral Therapy) resulted in a remarkable improvement in anti-HIV therapy, leading to a large reduction in HIV-associated morbity and mortality. HAART involves various combinations of nucleoside reverse transcriptase inhibitors (NRTIs), non-nucleoside reverse transcriptase inhibitors (NNRTIs) and protease inhibitors (PIs). Current guidelines for antiretroviral therapy recommend such triple combination therapy regimen even for initial treatment. However, none of the currently available drug therapies is capable of completely eradicating HIV. Even HAART can face the emergence of resistance, often due to non-adherence and non-persistence with antiretroviral therapy. In these cases HAART can be made effective again by replacing one of its components by one of another class. If applied correctly, treatment with HAART combinations can suppress the virus for many years, up to decades, to a level where it no longer can cause the outbreak of AIDS.

NRTIs are a basic component of HAART combinations. An NRTI that is frequently used in current combinations is tenofovir or its derivative tenofovir disoproxil fumarate (TDF), usually in combination with 3TC or with FTC. The use of tenofovir disoproxil fumarate, or of drug combinations containing tenofovir disoproxil fumarate, results in the selection of HIV that has a K65R mutation in reverse transcriptase.

A series of compounds that belong to a novel class of HIV RT inhibitors has been described^{1,2}. They differ from N(t)RTIs by chemical structure, absence of chain terminating properties, and lack of phosphorylation requirement, and from NNRTIs in terms of mechanism of action and binding pocket. Since they reversibly bind to the RT active site in competition with natural dNTP substrates, this class is referred to as Nucleotide-competing RT Inhibitors (NcRTIs).

It now has been found that NcRTIs show hypersusceptibilty towards the K65R mutation and therefore can be used to treat patients that are infected with HIV having this mutation. The present invention is aimed at using NcRTIs for treating patients that are infected with HIV having a K65R mutation in the viral genome.

### SUMMARY OF THE INVENTION

The present invention relates to a method for treating patients infected with HIV that has a K65R mutation in the viral genome encoding for reverse transcriptase, said method comprising administering an effective amount to said patients of an NcRTI. In another aspect there is provided a method for treating patients infected with HIV that has a K65R mutation in the viral genome encoding for reverse transcriptase, said method comprising administering an effective amount to said patients of a combination of HIV inhibitors, at least one of which is an NcRTI.

Or, in another aspect, the invention provides the use of an NcRTI for the manufacture of a medicament for treating patients infected with HIV that has that has a K65R mutation in the viral genome encoding for reverse transcriptase. In another aspect, the invention provides the use of a combination of HIV inhibitors, at least one of which is an NcRTI for the manufacture of a medicament for treating patients infected with HIV that has that has a K65R mutation in the viral genome encoding for reverse transcriptase.

### DESCRIPTION OF THE INVENTION

Nucleotide-competing reverse transcriptase inhibitors (NcRTI) bind to the active site of HIV reverse transcriptase (RT) in competition with the next incoming nucleotide. NcRTIs have been found to be ribonucleotide or pyrophosphate sensitive RT inhibitors and can be identified by running a test compound through an enzymatic RT inhibitory test with or without a nucleoside phosphate or a pyrophosphate being present and selecting those compounds that show an increase in inhibition of reverse transcriptase.

NcRTIs therefore can be found by a method comprising the steps:
a) providing a reaction well comprising at least one template for an HIV RT enzyme, at least one primer, at least one detectable dNTP substrate, at least one test compound; at least one RT enzyme, wherein said HIV RT enzyme incorporates the detectable dNTP substrate; and determining RT activity by measuring the amount of the detectable dNTP substrate incorporated into the template;
b) providing another reaction well comprising at least one template for an HIV RT enzyme, at least one primer, at least one detectable dNTP substrate, at least one test compound; at least one nucleoside phosphate or at least one pyrophosphate, at least one RT enzyme, wherein said HIV RT enzyme incorporates the detectable dNTP substrate; and determining RT activity by measuring the amount of the detectable dNTP substrate incorporated into the template;
c) comparing the RT activity obtained in step a) and in step b);
d) selecting the test compound wherein the RT inhibitory activity obtained in b) exceeds the RT inhibitory activity obtained in a);
   wherein the amount of the HIV RT inhibitor in steps a) and e) is the same and is such that an increase of RT activity is measurable.

NcRTs can also be identified as follows:
a) providing test compounds that are other than nucleoside triphosphates;
b) subjecting test compounds to a wild-type HIV virus replication test in cells;
c) subjecting test compounds to a NNRTI resistant HIV virus replication test in cells;
d) subjecting the test compounds to a kinetic reverse transcriptase enzymatic assay; and identifying the test compounds that are competitive towards the incorporated nucleotide in said assay;
   selecting the test compounds that are as well active in step b), are active in step c) and are identified as being competitive towards the incorporated nucleotide in step d).

NcRTIs in particular are those compounds that are as well competitive towards an incorporated nucleotide and that may be identified as described in the previous paragraph, as being ribonucleotide or pyrophosphate sensitive RT inhibitors, which may be identified by the methodology mentioned above.

NcRTIs have been described, for example, in WO 04/046163, WO 05/111034, WO 05/111035, WO 05/111047 and WO 05/111044. Combinations of the compounds of WO 04/046163 with certain HIV inhibitors have been described in WO 05/110411.

Interesting NcRTIs for use in the invention are those compounds having the formula:

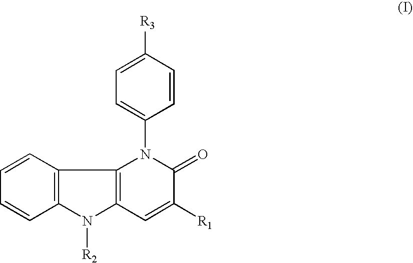

wherein
R₁ is cyano, methyloxycarbonyl, methylaminocarbonyl, ethyloxycarbonyl and ethylaminocarbonyl, more in particular wherein R₁ is cyano, ethyloxycarbonyl and ethylaminocarbonyl, even more in particular wherein R₁ is cyano.
R₂ is hydrogen, C₁₋₆alkyl optionally substituted with cyano, NR₄ₐR_{4b}, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxycarbonyl; in particular R₂ is C₁₋₆alkyl, hydrogen, C₂₋₆alkenyl;
R₃ is nitro, cyano, amino, halo, hydroxy, C₁₋₄alkyloxy, hydroxycarbonyl, aminocarbonyl, C₁₋₄alkyloxycarbonyl, mono- or di(C₁₋₄alkyl)aminocarbonyl, C₁₋₄alkylcarbonyl, or Het₁; in particular R₃ is nitro;
R₄ₐ and R_{4b} independently from each other are hydrogen, C₁₋₄alkyl or C₁₋₄alkyl substituted with amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl; in particular R₄ₐ and R_{4b} independently from each other are hydrogen, C₁₋₄alkyl;
Het₁ is furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, each optionally substituted with C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyano-C₁₋₄alkyl, mono- or di(C₁₋₄alkyl)amino, aminoC₁₋₄alkyl, mono- or di(C₁₋₄alkyl)-aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl; in particular Het₁ is furanyl, thienyl, pyrrolyl, oxazolyl, thiazolyl, imidazolyl, isoxazolyl, isothiazolyl, pyrazolyl, oxadiazolyl, thiadiazolyl, triazolyl, each optionally substituted with C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, as well as the pharmaceutically acceptable addition salts thereof.

Of interest are the compounds of formula (I) wherein R² is hydrogen, C₁₋₆alkyl optionally substituted with NR₄ₐR_{4b}, pyrrolidinyl, piperidinyl, homopiperidinyl, piperazinyl, 4-(C₁₋₄alkyl)-piperazinyl, morpholinyl, aryl, furanyl.

Of particular interest are the compounds of formula (I) wherein R² is C₁₋₆alkyl optionally substituted with NR₄ₐR_{4b}, pyrrolidinyl, piperidinyl, morpholinyl; and wherein R₄ₐ and R_{4b} independently from each other are hydrogen, C₁₋₄alkyl.

Also interesting for use in the present invention are compounds of formula (II)

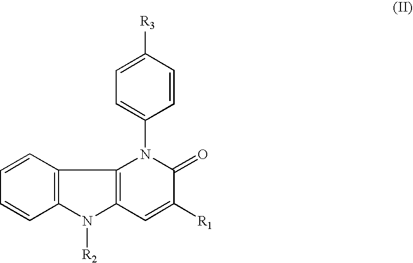

wherein R¹ is as R₁, R³ is as R₃ and
R² is C₁₋₆alkyl substituted with a radical selected from —NR^{5a}—C(═NR^{5b})—NR^{5c}R^{5d}, —NR^{5a}—C(═NR^{5e})—R^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R³, —NR⁹R¹⁰, a radical

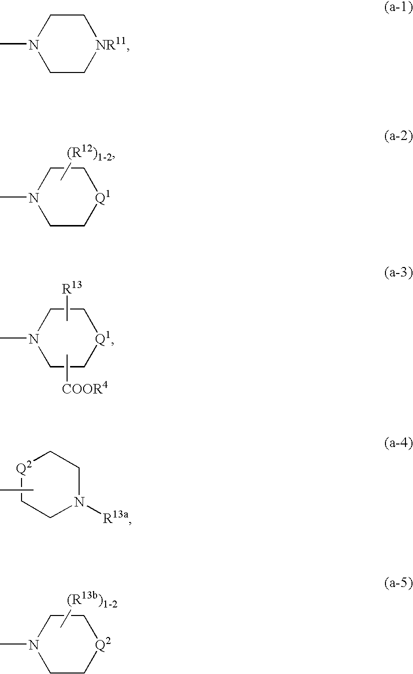

wherein
each Q¹ independently is a direct bond, —CH₂—, or —CH₂—CH₂—;
each Q² independently is O, S, SO or SO₂;
each R⁴ independently is hydrogen, C₁₋₄alkyl, arylC₁₋₄alkyl;
each R^{5a}, R^{5b},R^{5c}, R^{5d} independently is hydrogen, C₁₋₄alkyl or arylC₁₋₄alkyl;
each R^{5e}, R^{5f} independently is hydrogen, C₁₋₄alkyl or arylC₁₋₄alkyl, or R^{5e} and R^{5f}, taken together may form a bivalent alkanediyl radical of formula —CH₂—CH₂— or —CH₂—CH₂—CH₂—;
R⁶ is C₁₋₄alkyl, —N(R^{5a}R^{5b}), C₁₋₄alkyloxy, pyrrolidin-1-yl, piperidin-1-yl, homopiperidin-1-yl, piperazin-1-yl, 4-(C₁₋₄alkyl)-piperazin-1-yl, morpholin-4-yl-, thiomorpholin-4-yl-;
R⁷ is hydrogen, C₁₋₄alkyl, hydroxyC₁₋₄alkyl, C₁₋₄alkoxyC₁₋₄alkyl or C₁₋₄alkylcarbonyl-oxyC₁₋₄alkyl;
R⁸ is hydroxyC₁₋₄alkyl, C₁₋₄alkoxyC₁₋₄alkyl, C₁₋₄alkylcarbonyloxyC₁₋₄alkyl, aryl or arylC₁₋₄alkyl;
R⁹ is hydrogen or C₁₋₄alkyl;
R¹⁰ is Het₁, Het₂ or a radical

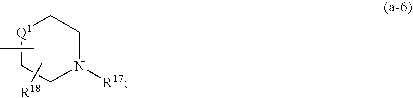

R¹¹ is aryl, arylC₁₋₄alkyl, formyl, C₁₋₄alkylcarbonyl, arylcarbonyl, arylC₁₋₄alkyl-carbonyl, C₁₋₄alkyloxycarbonyl, arylC₁₋₄alkyloxycarbonyl, R^{5a}R^{5b}N-carbonyl, hydroxyC₁₋₄alkyl, C₁₋₄alkyloxyC₁₋₄alkyl, arylC₁₋₄alkyloxyC₁₋₄alkyl, aryloxyC₁₋₄alkyl, Het₂;
each R¹² independently is hydroxy, C₁₋₄alkyl, arylC₁₋₄alkyl, C₁₋₄alkyloxy, arylC₁₋₄alkyloxy, oxo, spiro(C₂₋₄alkanedioxy), spiro(diC₁₋₄alkyloxy), —NR^{5a}R^{5b};
R¹³ is hydrogen, hydroxy, C₁₋₄alkyl, C₁₋₄alkyloxy, or arylC₁₋₄alkyloxy; or
R^{13a} is C₁₋₄alkyl, arylC₁₋₄alkyl, C₁₋₄alkyloxycarbonyl or arylC₁₋₄alkyloxycarbonyl;
each R^{13b} is hydrogen or C₁₋₄alkyl; or R² is
a radical of formula:

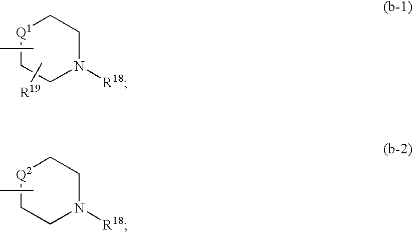

—CₚH₂ₚ—CH(OR¹⁴)—C_{q}H_{2q}—R¹⁵ (b-3);

—CH₂—CH₂—(O—CH₂—CH₂)ₘ—OR¹⁴ (b-4);

—CH₂—CH₂—(O—CH₂—CH₂)ₘ—NR^{17a}R^{17b} (b-5);

wherein in radical (b-3) one of the hydrogen atoms in —CₚH₂ₚ— and one of the hydrogen atoms in —CH(OR¹⁴)—C_{q}H_{2q}—, that is not part of R¹⁴, may be replaced by a direct bond or a C₁₋₄alkanediyl group;
p is 1, 2 or 3;
q is 0, 1, 2 or 3;
each m independently is 1 to 10;
each R¹⁴ independently is hydrogen, C₁₋₄alkyl, aryl C₁₋₄alkyl, aryl, C₁₋₄alkylcarbonyl, —SO₃H, —PO₃H₂;
R¹⁵ is cyano, NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, 4-(C₁₋₄alkyloxycarbonyl)-piperazinyl, morpholinyl, thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxy-carbonyl, C₁₋₄alkylcarbonyl, N(R^{16a}R^{16b})carbonyl, C₁₋₄alkyloxycarbonyl, pyrrolidin-1-ylcarbonyl, piperidin-1-ylcarbonyl, homopiperidin-1-ylcarbonyl, piperazin-1-ylcarbonyl, 4-(C₁₋₄alkyl)-piperazin-1-ylcarbonyl, morpholin-1-yl-carbonyl, thiomorpholin-1-yl-carbonyl, 1-oxothiomorpholin-1-ylcarbonyl and 1,1-dioxo-thiomorpholin-1-ylcarbonyl; or R¹⁵ may additionally be aryl substituted with a radical —COOR⁴; or a radical selected from —NR^{5a}—C(═NR^{5b})—NR^{5c}R^{5d}, —NR^{5a}—C(═NR^{5e})—R^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R⁸, —NR⁹R¹⁰, a radical (a-1), (a-2), (a-3), (a-4) or (a-5); wherein R⁴, R^{5a}, R^{5b}, R^{5c}, R^{5d}, R⁶, R⁷, R⁸, R⁹, R¹⁰, and the radicals (a-1), (a-2), (a-3), (a-4), (a-5), independently are as defined above;
R^{16a} and R^{16b} independently from one another are hydrogen, C₁₋₆alkyl or C₁₋₆alkyl substituted with a substituent selected from the group consisting of amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl and aryl;
R^{17a} and R^{17b} independently from one another are hydrogen, C₁₋₄alkyl or arylC₁₋₄alkyl; or R^{17a} and R^{17b} together with the nitrogen atom to which they are attached form a pyrrolidinyl, piperidinyl, homopiperidinyl, morpholinyl, thiomorpholinyl, piperazinyl, 4-C₁₋₄alkyl-piperazinyl, 4-(C₁₋₄alkylcarbonyl)-piperazinyl, 4-(C₁₋₄alkyloxycarbonyl)-piperazinyl ring;
   each R¹⁸ independently is hydrogen, C₁₋₄alkyl, arylC₁₋₄alkyl, C₁₋₄alkylcarbonyl or C₁₋₄alkyloxycarbonyl;
   R¹⁹ is hydrogen, hydroxy, C₁₋₄alkyl or a radical —COOR⁴; R⁴ is hydrogen or C₁₋₄alkyl; as well as the pharmaceutically acceptable addition salts thereof.

Also interesting for use in the invention are the following compounds:

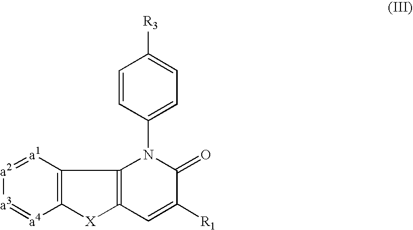

wherein R¹, R³ and R² are as specified above for (I) or for (II) and −a¹=a²−a³=a⁴-represents a bivalent radical of formula

—CH═CH—CH═CH— (c-1);

wherein one, two, three or four of the hydrogen atoms in (c-1) is replaced by a radical C₁₋₆alkyl, C₁₋₄alkoxy, halo, hydroxy, (R^{5g})(R^{5h})N—(C₁₋₄alkanediyl)-O—, (R⁷)(R⁸)N—(C₁₋₄alkanediyl)-O—, trifluoromethyl, cyano, a radical —COOR⁴, (R^{5a})(R^{5b})N-carbonyl, formyl, C₁₋₆alkylcarbonyl, nitro, hydroxyC₁₋₆alkyl, C₁₋₄alkoxyC₁₋₆alkyl, (R⁴OOC)—C₁₋₆alkyl, a radical —N(R^{5a})(R^{5b}), a radical

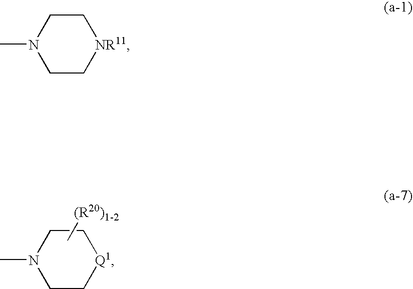

morpholinyl, thiomorpholinyl, (R^{5g})(R^{5h})N—(C₁₋₄alkanediyl)-N(R^{5c})—, (R⁷)(R⁸)N—(C₁₋₄alkanediyl)-N(R^{5c})—, C₁₋₆alkyl-carbonylamino, C₁₋₆alkyloxycarbonylamino, trifluoroacetylamino, C₁₋₆alkylsulfonyl-amino, (R^{5a})(R^{5b})N—C₁₋₄alkyl; aryl;
R^{5g} and R^{5h} independently are hydrogen or C₁₋₄alkyl; R^{5a} and R^{5c} independently are hydrogen or C₁₋₄alkyl; Q¹ and R¹¹ are as defined above; as well as the pharmaceutically acceptable addition salts thereof.

An interesting subgroup of the compounds of formula (I), (II) or (III) comprises those compounds, wherein R₃ or R³ is nitro.

In any of the above compounds represented by (I), (II) or (III) the phenyl moiety substituted with R₃ or R³ may be replaced by furyl, thienyl, pyridyl, pyrimidinyl, benzofuryl, benzo-thienyl, indolyl, imidazopyridyl, purinyl, optionally substituted with one or two substituents selected from halo, cyano, C₁₋₆alkyl, CF₃, —COOR⁴, (R^{5a})(R^{5b})N-carbonyl, hydroxy, C₁₋₆alkyloxy, C₁₋₆alkylthio, C₁₋₆alkylsulfonyl; or the phenyl moiety substituted with R₃ or R³ may be replaced by furyl, thienyl, pyridyl, indolyl, imidazopyridyl, optionally substituted with one or two substituents selected from halo, cyano, C₁₋₆alkyl, CF₃, —COOR⁴, (R^{5a})(R^{5b})N-carbonyl, C₁₋₆alkyloxy, C₁₋₆alkylthio, C₁₋₆alkylsulfonyl; or the phenyl moiety substituted with R₃ or R³ may be replaced by halopyridyl, in particular by 6-chloro-4-pyridyl.

The term “C₁₋₄alkyl” as a group or part of a group defines straight and branched chained saturated hydrocarbon radicals having from 1 to 4 carbon atoms, such as, for example, methyl, ethyl, propyl, butyl, 2-methyl-propyl and the like. The term “C₁₋₆alkyl” as a group or part of a group defines straight and branched chained saturated hydrocarbon radicals having from 1 to 6 carbon atoms such as, for example, the groups defined for C₁₋₄alkyl and pentyl, hexyl, 2-methylbutyl, 3-methylpentyl and the like.

The term “C₂₋₆alkyl” as a group or part of a group defines straight and branched chained saturated hydrocarbon radicals having from 2 to 6 carbon atoms such as for example, ethyl, propyl, butyl, 2-methyl-propyl, pentyl, hexyl, 2-methylbutyl, 3-methylpentyl and the like.

The term “C₁₋₁₀alkyl” as a group or part of a group defines straight and branched chained saturated hydrocarbon radicals having from 1 to 10 carbon atoms such as, for example, the groups defined for C₁₋₆alkyl and heptyl, octyl, nonyl, decyl and the like. The term C₂₋₆alkenyl as a group or part of a group defines straight and branched chained hydrocarbon radicals having saturated carbon-carbon bonds and at least one double bond, and having from 2 to 6 carbon atoms, such as, for example, ethenyl, prop-1-enyl, but-1-enyl, but-2-enyl, pent-1-enyl, pent-2-enyl, hex-1-enyl, hex-2-enyl, hex-3-enyl, 1-methyl-pent-2-enyl and the like.

The term “C₂₋₁₀alkenyl” as a group or part of a group defines straight and branched chained hydrocarbon radicals having saturated carbon-carbon bonds and at least one double bond, and having from 2 to 10 carbon atoms, such as, for example, the groups of C₂₋₆alkenyl and hept-1-enyl, hept-2-enyl, hept-3-enyl, oct-1-enyl, oct-2-enyl, oct-3-enyl, non-1-enyl, non-2-enyl, non-3-enyl, non-4-enyl, dec-1-enyl, dec-2-enyl, dec-3-enyl, dec-4-enyl, 1-methyl-pent-2-enyl and the like. The term C₃₋₇cycloalkyl is generic to cyclopropyl, cyclobutyl, cyclopentyl, cyclohexyl and cycloheptyl.

The term “halo” is generic to fluoro, chloro, bromo or iodo.

Aryl is phenyl optionally substituted with one, two or three substituents independently selected from C₁₋₆alkyl, C₁₋₄alkoxy, halo, hydroxy, amino, trifluoromethyl, mono- and di-C₁₋₆alkylamino, nitro, cyano, carboxyl, C₁₋₄alkoxycarbonyl; in particular aryl is phenyl optionally substituted with one, two or three substituents independently selected from C₁₋₆alkyl, C₁₋₄alkoxy, halo, hydroxy, amino, nitro, cyano.

It should be noted that different isomers of the various heterocycles may exist within the definitions as used throughout the specification. For example, oxadiazolyl may be 1,2,4-oxadiazolyl or 1,3,4-oxadiazolyl or 1,2,3-oxadiazolyl; likewise for thiadiazolyl which may be 1,2,4-thiadiazolyl or 1,3,4-thiadiazolyl or 1,2,3-thiadiazolyl; pyrrolyl may be 1H-pyrrolyl or 2H-pyrrolyl.

It should also be noted that the radical positions on any molecular moiety used in the definitions may be anywhere on such moiety as long as it is chemically stable. For instance pyridyl includes 2-pyridyl, 3-pyridyl and 4-pyridyl; pentyl includes 1-pentyl, 2-pentyl and 3-pentyl.

Examples of compounds for use in the invention are:

1-(4-Nitro-phenyl)-2-oxo-2,5-dihydro-1H-pyrido[3,2-b]indole-
3-carbonitrile;
5-Methyl-1-(4-nitro-phenyl)-2-oxo-2,5-dihydro-1H-pyrido[3,2-
b]indole-3-carbonitrile;
5-Isobutyl-1-(4-nitro-phenyl)-2-oxo-2,5-dihydro-1H-pyrido[3,2-
b]indole-3-carbonitrile;
5-Allyl-1-(4-nitro-phenyl)-2-oxo-2,5-dihydro-1H-pyrido[3,2-
b]indole-3-carbonitrile;
5-Butyl-1-(4-nitro-phenyl)-2-oxo-2,5-dihydro-1H-pyrido[3,2-
b]indole-3-carbonitrile;
5-Ethyl-1-(4-nitro-phenyl)-2-oxo-2,5-dihydro-1H-pyrido[3,2-
b]indole-3-carbonitrile;
5-(2-Morpholin-4-yl-ethyl)-1-(4-nitro-phenyl)-2-oxo-2,5-dihydro-
1H-pyrido[3,2-b]-indole-3-carbonitrile;
5-Methyl-1-(4-nitro-phenyl)-1,5-dihydro-pyrido[3,2-b]indol-2-one;
5-But-3-enyl-1-(4-nitro-phenyl)-2-oxo-2,5-dihydro-1H-
pyrido[3,2-b]indole-3-carbonitrile;
1-(4-Nitro-phenyl)-2-oxo-5-(2-pyrrolidin-1-yl-ethyl)-2,5-dihydro-
1H-pyrido[3,2-b]-indole-3-carbonitrile;
1-(4-Nitro-phenyl)-2-oxo-5-(2-piperidin-1-yl-ethyl)-2,5-dihydro-
1H-pyrido[3,2-b]-indole-3-carbonitrile;
5-(3-Dimethylamino-propyl)-1-(4-nitro-phenyl)-2-oxo-2,5-dihydro-
1H-pyrido[3,2-b]-indole-3-carbonitrile;
3-Bromo-5-methyl-1-(4-nitro-phenyl)-1,5-dihydro-pyrido[3,2-
b]indol-2-one
5-Methyl-1-(3-nitro-phenyl)-2-oxo-2,5-dihydro-1H-pyrido[3,2-
b]indole-3-carbonitrile;
1-(4-Nitro-phenyl)-2-oxo-5-(3-piperidin-1-yl-propyl)-2,5-dihydro-
1H-pyrido[3,2-b]-indole-3-carbonitrile;
5-(4-Morpholin-4-yl-butyl)-1-(4-nitro-phenyl)-2-oxo-2,5-dihydro-
1H-pyrido[3,2-b]-indole-3-carbonitrile;
1-(4-Nitro-phenyl)-2-oxo-5-(4-pyrrolidin-1-yl-butyl)-2,5-dihydro-
1H-pyrido[3,2-b]-indole-3-carbonitrile;
5-[3-(4-Methyl-piperazin-1-yl)-propyl]-1-(4-nitro-phenyl)-2-
oxo-2,5-dihydro-1H-pyrido[3,2-b]indole-3-carbonitrile;
5-Cyanomethyl-1-(4-nitro-phenyl)-2-oxo-2,5-dihydro-1H-
pyrido[3,2-b]indole-3-carbonitrile;
5-(3-Morpholin-4-yl-propyl)-1-(4-nitro-phenyl)-2-oxo-2,5-dihydro-
1H-pyrido[3,2-b]-indole-3-carbonitrile;
1-(4-Nitro-phenyl)-2-oxo-5-(4-piperidin-1-yl-butyl)-2,5-dihydro-
1H-pyrido[3,2-b]-indole-3-carbonitrile;
5-(4-Dimethylamino-butyl)-1-(4-nitro-phenyl)-2-oxo-2,5-dihydro-
1H-pyrido[3,2-b]-indole-3-carbonitrile;
1-(4-Nitro-phenyl)-2-oxo-5-pyridin-4-ylmethyl-2,5-dihydro-1H-
pyrido[3,2-b]indole-3-carbonitrile;
3-(5-tert-Butyl-[1,2,4]oxadiazol-3-yl)-5-methyl-1-(4-nitro-
phenyl)-1,5-dihydro-pyrido[3,2-b]indol-2-one; or
5-Methyl-1-(4-nitro-phenyl)-3-(5-trifluoromethyl-[1,2,4]oxadiazol-
3-yl)-1,5-dihydro-pyrido[3,2-b]indol-2-one, salts.
Particular compounds are:
5-(2-Morpholin-4-yl-ethyl)-1-(4-nitro-phenyl)-2-oxo-2,5-
dihydro-1H-pyrido[3,2-b]-indole-3-carbonitrile;
1-(4-Nitro-phenyl)-2-oxo-5-(2-piperidin-1-yl-ethyl)-2,5-
dihydro-1H-pyrido[3,2-b]-indole-3-carbonitrile;
1-(4-Nitro-phenyl)-2-oxo-5-(2-pyrrolidin-1-yl-ethyl)-2,5-
dihydro-1H-pyrido[3,2-b]-indole-3-carbonitrile.

A compound of particular interest is:

5-Methyl-1-(4-nitro-phenyl)-2-oxo-2,5-dihydro-1H-pyrido[3,2-b]indole-3-carbonitrile; hereafter referred to as “NcRTI-1”, which compound is represented by the following chemical structure:

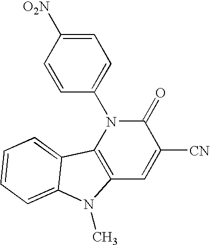

Further compounds of interest are:
1-(4-Nitro-phenyl)-2-oxo-1,2-dihydro-benzo[4,5]furo[3,2-b]pyridine-3-carbonitrile,
5-(2-Hydroxy-3-piperidin-1-yl-propyl)-1-(4-nitro-phenyl)-2-oxo-2,5-dihydro-1H-pyrido[3,2-b]indole-3-carbonitrile,
5-(3-Diethylamino-2-hydroxy-propyl)-1-(4-nitro-phenyl)-2-oxo-2,5-dihydro-1H-pyrido[3,2-b]indole-3-carbonitrile,
5-[2-(2-Methoxy-ethoxy)-ethyl]-1-(4-nitro-phenyl)-2-oxo-2,5-dihydro-1H-pyrido-[3,2-b]indole-3-carbonitrile, and especially
5-(2-Hydroxy-3-pyrrolidin-1-yl-propyl)-1-(4-nitro-phenyl)-2-oxo-2,5-dihydro-1H-pyrido[3,2-b]indole-3-carbonitrile; or
5-(2-Hydroxy-3-morpholin-4-yl-propyl)-1-(4-nitro-phenyl)-2-oxo-2,5-dihydro-1H-pyrido[3,2-b]indole-3-carbonitrile.

Further compounds of interest are:
8-bromo-5-methyl-1-(4-nitro-phenyl)-2-oxo-2,5-dihydro-1H-pyrido[3,2-b]indole-3-carbonitrile,
8-morpholin-5-methyl-1-(4-nitro-phenyl)-2-oxo-2,5-dihydro-1H-pyrido[3,2-b]indole-3-carbonitrile,
8-hydroxy-5-methyl-1-(4-nitro-phenyl)-2-oxo-2,5-dihydro-1H-pyrido[3,2-b]indole-3-carbonitrile,
8-hydroxy-1-(4-nitro-phenyl)-2-oxo-2,5-dihydro-1H-pyrido[3,2-b]indole-3-carbonitrile,
8-methoxy-5-methyl-1-(4-nitro-phenyl)-2-oxo-2,5-dihydro-1H-pyrido[3,2-b]indole-3-carbonitrile,
8-[(3-Dimethylamino-propyl)-methylamino]-5-methyl-1-(4-nitro-phenyl)-2-oxo-2,5-dihydro-1H-pyrido[3,2-b]indole-3-carbonitrile, or
8-[(3-Dimethylamino-propyl)-oxy]-5-methyl-1-(4-nitro-phenyl)-2-oxo-2,5-dihydro-1H-pyrido[3,2-b]indole-3-carbonitrile.

Further compounds of interest are:
8-Methoxy-5-methyl-1-(6-methyl-pyridin-3-yl)-2-oxo-2,5-dihydro-1H-pyrido-[3,2-b]indole-3-carbonitrile;
1-(2,8-Dimethyl-imidazo[1,2-a]pyridin-6-yl)-5-methyl-2-oxo-2,5-dihydro-1H-pyrido[3,2-b]indole-3-carbonitrile;
1-(6-Chloro-5-methyl-pyridin-3-yl)-5-methyl-2-oxo-2,5-dihydro-1H-pyrido[3,2-b]-indole-3-carbonitrile;
1-(6-Chloro-pyridin-3-yl)-8-hydroxy-5-methyl-2-oxo-2,5-dihydro-1H-pyrido[3,2-b]-indole-3-carbonitrile;
1-(6-Chloropyridin-3-yl)-5-methyl-2-oxo-2,5-dihydro-1H-pyrido[3,2-b]indole-3-carbonitrile;
5-Methyl-1-(2-methyl-imidazo[1,2-a]pyridin-6-yl)-2-oxo-2,5-dihydro-1H-pyrido-[3,2-b]indole-3-carbonitrile; or
5-Methyl-1-(6-methyl-pyridin-3-yl)-2-oxo-2,5-dihydro-1H-pyrido[3,2-b]indole-3-carbonitrile.

Compounds of particular interest are:
1-(4-Acetyl-phenyl)-3-methyl-2-oxo-1,2-dihydro-benzo[4,5]furo[3,2-b]pyridine-4-carbonitrile;
4-Methyl-1-(4-nitrophenyl)-2-oxo-1,2-dihydro-furo[3,2-b; 4,5-b′]dipyridine-3-carbonitrile;
7-Methoxy-4-methyl-1-(4-nitrophenyl)-2-oxo-1,2-dihydro-benzo[4,5]furo[3,2-b]-pyridine-3-carbonitrile;
7-Hydroxy-4-methyl-1-(4-nitrophenyl)-2-oxo-1,2-dihydro-benzo[4,5]furo[3,2-b]-pyridine-3-carbonitrile.

The above-mentioned compounds may be used in free form or as a pharmaceutically acceptable addition salt form wherein the salts may be derived from acids such as, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butandioic acid), maleic, fumaric, malic (i.e. hydroxyl-butandioic acid), tartaric, citric, methanesulfonic, ethanesulfonic, benzenesulfonic, p-toluenesulfonic, cyclamic, salicylic, p-aminosalicylic, pamoic and the like acids. A particular group of compounds for use in this invention are the pharmaceutically acceptable addition salts of the compounds listed above by their chemical name.

Where applicable, the above compounds or their salts may be used in the form of racemates, stereoisomers or stereoisomeric mixtures.

The NcRTI that is administered, either alone or in a combination with one or more other HIV inhibitory agents, preferably is formulated into a suitable pharmaceutical formulation. Suitable formulations have been described in the references cited in the previous paragraph. The NcRTI can be administered as such, or in salt form; it can further be administered in the form of a stereoisomer or stereoisomeric mixture. It can also be administered as a pro-drug. Salts, stereoisomers and stereoisomeric mixtures and pro-drugs have also been described in the references cited in the previous paragraph.

In a further aspect, the present invention provides a method for treating patients infected with HIV that have been treated with tenofovir (or a derivative such as TDF) and/or 3TC, said method comprising administering an effective amount to said patients of an NcRTI. Or alternatively, the invention provides the use of an NcRTI for the manufacture of a medicament for treating patients infected with HIV that have been treated with tenofovir (or a derivative such as TDF) and/or 3TC.

### EXPLANATION OF THE FIGURES

FIG. 1: A random set of over 1700 clinical HIV-1 isolates was categorized into 6 groups according to their genotype. For each group the median FC for NcRTI-1, the interquartile range, and the number of viruses is shown. WT indicates the group of viruses without mutations at position 184, 115 or 65.

FIG. 2: In vitro selection experiment with NcRTI-1 starting from HIV-1 IIIB. Each dot represents virus breakthrough at the indicated NcRTI-1 concentration. Mutational patterns found in the harvested virus population are shown.

### EXAMPLE 1

The susceptibility of over 6000 recent clinical HIV-1 isolates for NcRTI-1 and different N(t)RTIs and NNRTIs was assessed.

82% of the profiled clinical isolates remained susceptible for NcRTI-1 (fold change in EC₅₀ (FC)<4), 15.7% of the population showed a reduced susceptibility (FC4-10) while only 2.3% showed resistance (FC>10). No cross-resistance was observed between NcRTI-1 and the NNRTIs EFV or NVP, neither with the N(t)RTIs ZDV, TDF, and ABC. Only with the N(t)RTIs 3TC and FTC, limited cross-resistance could be detected (Pearson Correlation Coefficient=0.56) (Table 1).

TABLE 1
Resistance correlation between different RT inhibitors
is shown as the correlation coefficient calculated by
linear regression of a cross-correlation plot.

| | 3TC | ZDV | TDF | ABC | NVP | EFV | NcRTI-1 |
|---|---|---|---|---|---|---|---|
| 3TC | 1.0 | 0.28 | 0.20 | 0.60 | 0.33 | 0.28 | 0.56 |
| ZDV | | 1.0 | 0.64 | 0.58 | 0.30 | 0.29 | 0.17 |
| TDF | | | 1.0 | 0.45 | 0.29 | 0.27 | 0.06 |
| ABC | | | | 1.0 | 0.34 | 0.33 | 0.32 |
| NVP | | | | | 1.0 | 0.82 | 0.20 |
| EFV | | | | | | 1.0 | 0.16 |
| NcRTI-1 | | | | | | | 1.0 |

| | | | |
|---|---|---|---|
| Correlation | <0.4 | 0.4-0.7 | >0.7 |
| coefficient | | | |

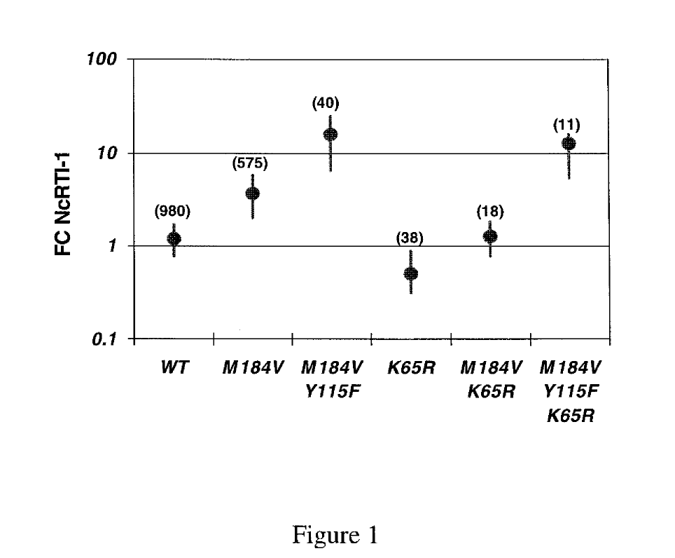

Analysis of the genotype of a random set of more than 1700 of the 6000 tested clinical isolates (FIG. 1) showed that the combination of mutations M184V+Y115F correlated most with resistance to NcRTI-1 (FC=75). Given that Met¹⁸⁴ and Tyr¹¹⁵ are part of the HIV RT active site, this finding underscores the mechanistic model for NcRTIs.

Analysis also indicated that the K65R mutation not only is associated with hypersusceptibility to NcRTI-1 but also reverses M184V-induced resistance of HIV-1 for NcRTI-1. These findings were confirmed in site-directed mutant (SDM) strains.

This reciprocity between the K65R and M184V mutation is unparalleled among RT inhibitors. When replicating wild-type HIV-1 in the presence of NcRTI-1, M184V+Y115F were selected. In the presence of both NcRTI-1 and tenofovir, NcRTI-1 prevents the selection of K65R. NcRTI-1 activity is not affected by the presence of NNRTI resistance mutations, nor by the presence of the major N(t)RTI induced, multi-drug resistant mutation patterns: the thymidine-associated mutations (TAMs), the T69 insertion complex, and the Q151M complex.

Findings with clinical isolates were confirmed in site-directed mutant (SDM) strains (Table 2):

TABLE 2
Fold changes for HIV-1 strains with mutations in the RT gene were generated by site-directed
mutagenesis. The susceptibility for NcRTI-1 and other RT inhibitors is shown. Number of
measurements and interquartile ranges are indicated between brackets (n; Q1-Q3).

| | NcRTI-1 | 3TC | ZDV | TDF | NVP |
|---|---|---|---|---|---|
| Y115F M184V | 75 | >100 | 0.5 | 0.9 | 2.3 |
| | (2; 45-120) | (2) | (5; 0.45-1) | (5; 0.62-1.2) | (3; 2-2.7) |
| M184V | 5.0 | >100 | 1.0 | 0.8 | 2.3 |
| | (18; 3.8-6.5) | (19) | (24; 0.61-1.2) | (18; 0.44-1) | (16; 1.2-3.1) |
| Y115F | 7.9 | 0.7 | 0.9 | 1.0 | 2.9 |
| | (2; 6.3-14) | (4; 0.73-1.1) | (6; 0.67-1.1) | (6; 0.65-1.4) | (4; 2.3-5.3) |
| K065R | 0.5 | >20 | 2.0 | 6.0 | 3.4 |
| | (3; 0.29-0.57) | (2) | (4; 1.9-2.2) | (1) | (3; 2.4-3.7) |
| K065R M184V | 0.9 | >100 | 1.1 | 2.3 | 1.2 |
| | (6; 0.85-0.93) | (7) | (8; 0.87-1.3) | (8; 1.7-2.7) | (7; 1.1-1.5) |
| M041L D067N | 3.8 | 8.1 | 67 | 3.3 | 2.8 |
| K070R T215Y | (6; 1.9-3.8) | (4; 7.8-8.4) | (6; 22-75) | (6; 1.8-4.3) | (4; 2.5-3) |
| T069S ins069- | 0.9 | 9.5 | 53 | 2.3 | 1.1 |
| 070S-S L210W | (3; 0.84-1.1) | (2; 7.2-13) | (4; 36-68) | (2; 2.2-2.5) | (3; 0.65-1.5) |
| T215Y | | | | | |
| A62V V75I F77L | 1.0 | ND | >100 | 3.7 | ND |
| F116Y Q151M | (5; 0.88-1.2) | | (5) | (5; 3.4-6.7) | |
| Y181C | 2.3 | 1.1 | 0.6 | 0.7 | >100 |
| | (15; 1.3-5.4) | (62; 0.79-1.7) | (164; 0.4-1.1) | (105; 0.52-1) | (70) |
| K103N | 1.9 | 1.1 | 1.1 | 0.6 | 50 |
| | (3; 1.3-2) | (52; 0.86-1.5) | (111; 0.76-1.7) | (55; 0.5-0.85) | (8; 29-128) |
| K103N Y181C | 2.1 | 0.9 | 0.7 | 0.5 | >100 |
| | (147; 1.3-2.9) | (308; 0.71-1.2) | (538; 0.32-1.2) | (209; 0.28-0.78) | (6) |
| L100I K103N E138G | 2.5 | 0.7 | 0.4 | 0.4 | >100 |
| V179I Y181C | (150; 1.9-3.3) | (270; 0.45-0.85) | (430; 0.25-0.69) | (147; 0.32-0.6) | (5) |
| F227C L234I | 2.0 | 1.1 | 0.3 | 0.6 | 31 |
| | (5; 1.5-2.2) | (52; 0.94-1.4) | (118; 0.2-0.43) | (61; 0.39-0.78) | (9; 11-39) |
| Y188L | 1.7 | 1.9 | 0.7 | 0.7 | >100 |
| | (4; 1.6-1.8) | (51; 1.3-2.6) | (115; 0.42-1.1) | (57; 0.5-0.93) | (6;) |
| L100I K103N | 2.6 | 0.5 | 0.3 | 0.3 | >100 |
| | (5; 1.9-3.2) | (48; 0.3-0.68) | (147; 0.22-0.46) | (91; 0.24-0.44) | (7) |
| K101E K103N | 2.1 | 1.3 | 1.5 | 0.5 | >100 |
| | (5; 1.5-2.1) | (235; 0.94-1.8) | (302; 0.87-2.3) | (58; 0.4-0.75) | (5) |
| HIV-2 (ROD) | 14 | 0.6 | 0.1 | 1.0 | >100 |
| | (10; 10-17) | (29; 0.46-0.94) | (36; 0.031-0.12) | (10; 0.87-1.7) | (3) |

### EXAMPLE 2

Recombinant HIV-1 viruses, derived from clinical samples, were constructed by cotransfection of MT4 cells with sample derived viral protease and RT coding sequences and an HIV-1 HXB2-derived proviral clone deleted in the protease and RT coding region².

Site-directed mutant RT coding sequences were generated from a pGEM vector containing the HIV-1 clone HXB2 protease and RT coding sequence by using a QuikChange™ site-directed mutagenesis kit, and HPLC-purified primers. Plasmids were sequenced to confirm that they contained the desired mutations. Mutant viruses were created by recombination of the mutant protease-RT sequence with a protease-RT deleted HIV-1 HXB2 proviral clone².

To in vitro select viral strains resistant to RT inhibitors, MT4-LTR-EGFP cells were infected in the presence of the inhibitor (at 2 or 3 times EC₅₀). Cultures were passaged every 3 to 4 days at the same concentration of inhibitor, until full virus breakthrough. At that time, virus was harvested and used for a new round of selection at a higher compound concentration. At each breakthrough, the harvested virus was genotyped to identify acquired mutations. For several RT inhibitors, alone or in combination, the mutations selected when replicating wild type HIV-1 (IIIB) in their presence, were determined (see Table 3).

TABLE 3
In vitro_selection experiments were performed by replicating HIV-1
IIIB in the presence of one or two RT inhibitors. The table indicates
the number of passages, the final compound concentration and the
mutations present in the harvested virus population.

| | | Conc | |
|---|---|---|---|
| Compound(s) | Passage | (μM) | Selected RT Mutations |
| 3TC | 10 | 128 | M184V |
| 3TC | 4 | 8 | M184I |
| FTC | 6 | 4 | M184I |
| FTC | 5 | 4 | M184V |
| FTC | 6 | 4 | M184I |
| TDF | 10 | 40 | V179I K065R |
| TDF | 7 | 40 | K065R |
| TDF | 23 | 80 | K065R |
| NcRTI-1 | 10 | 2.4 | Y115F M184V |
| TDF/3TC | 12 | 20/16 | K065R E248K |
| TDF/3TC | 9 | 10/8 | K065R |
| TDF/FTC | 9 | 20/2 | K065R |
| TDF/FTC | 13 | 20/2 | K070E |
| TDF/FTC | 10 | 10/1 | K065R |
| TDF/NcRTI-1 | 57 | 40/0.6 | M041L K070N L193I |
| TDF/NcRTI-1 | 55 | 40/0.6 | M041L K070E Y181C N348I |
| TDF/NcRTI-1 | 21 | 20/0.3 | K070E |
| TDF/EFV | 17 | 40/0.008 | K065R K103N |

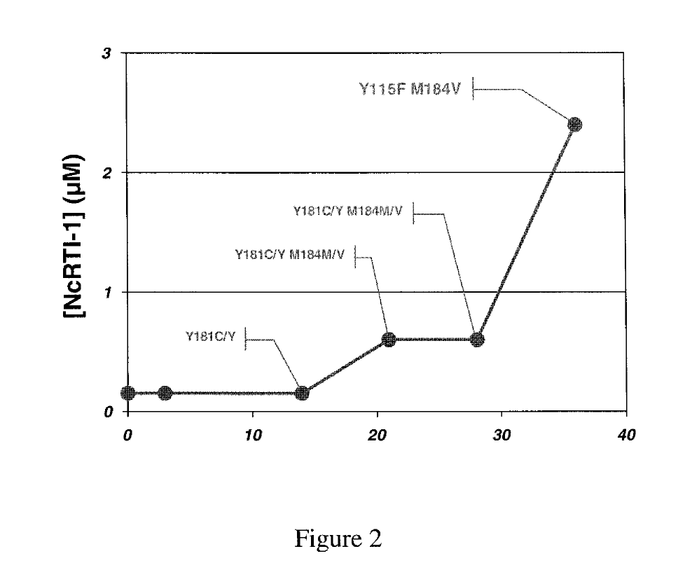

These results demonstrate that while the single mutations M184V and Y115F showed a moderate fold change of 5.0 and 7.9, respectively, the combination resulted in a FC of 75. When NcRTI-1 was present as sole inhibitor, wild type HIV-1 IIIB acquired mutations M184V and Y115F, in line with the results obtained with clinical isolates and SDMs (FIG. 2).

The presence of the K65R point mutation causes an increased susceptibility of HIV-1 for NcRTI-1 (FC=0.46), and reverses M184V-induced reduction of NcRTI sensitivity (FC from 5.0 to 0.89).

NcRTI-1 remained active on a wide variety of strains containing NNRTI resistance associated mutations. The major N(t)RTI induced, multi-drug resistant mutation patterns (TAMs, T69 insertion complex and Q151M complex) do not affect NcRTI-1 activity.

When 3TC or FTC was combined with TDF, the K65R mutation was selected, in accordance with previous findings³. Combining NcRTI-1 with TDF did not result in selection of the K65R mutation, even after prolonged exposure (190 days). Instead, the virus acquired the K70E or K70N mutation.

### CONCLUSIONS

From the above experiments, the following conclusions can be drawn. Mutational patterns associated with reduced and increased susceptibility to NcRTIs are different from patterns associated with currently used RT inhibitors. Only limited cross-resistance with 3TC was detected.

The combination of RT-active site mutations M184V+Y115F correlated most significantly with resistance to NcRTI-1 (FC=75). This combination is also selected in vitro when replicating HIV-1 IIIB in the presence of increasing concentrations of NcRTI-1.

Unlike 3TC or FTC, where M184V causes complete resistance (FC>100), the effect of M184V on NcRTI-1 susceptibility is limited (FC=5.0).

The K65R mutation causes hypersusceptibility to NcRTI-1. In addition, presence of K65R reverses the M184V-induced reduction of NcRTI sensitivity. This reciprocity between the K65R and M184V mutation is unparalleled among RT inhibitors.

When combined with TDF, NcRTI-1 prevents the selection of K65R by TDF. These experiments showed that the viruses acquired K70E, a mutation previously suggested as an alternative pathway of TDF resistance⁴.

The present invention also relates to a method for treating patients infected with HIV that has developed resistance towards an NcRTI, said method comprising administering an effective amount to said patients of tenofovir or of a tenofovir derivative, in particular tenofovir disoproxil fumarate (TDF). In particular said HIV that has developed resistance towards an NcRTI shows a fold change of at least 10, or at least 20, or at least about 50, or at least about 75. More in particular said HIV that has developed resistance towards an NcRTI has a M184V or Y115F, or a double M184V or Y115F mutation in the viral genome encoding for reverse transcriptase. This method is useful in treating patients that have been pre-treated with an NcRTI or NcRTI containing combination and have developed resistance towards the NcRTI, as indicated by the fold change. These patients can be treated with an effective amount to said patients of tenofovir or of a tenofovir derivative, in particular tenofovir disoproxil fumarate (TDF). In an alternative aspect, this invention concerns the use of tenofovir or of a tenofovir derivative, in particular tenofovir disoproxil fumarate (TDF) for the manufacture of a medicament for treating infected with HIV that has developed resistance towards an NcRTI, in particular with HIV that has developed resistance towards an NcRTI showing a fold change of at least 10, or at least 20, or at least about 50, or at least about 75; or more in particular with HIV that has developed resistance towards an NcRTI has a M184V or Y115F, or a double M184V or Y115F mutation in the viral genome encoding for reverse transcriptase.

All references mentioned in this specification are incorporated herein in their entirety.

### REFERENCES

1 Jochmans, D., Kesteleyn, B., Marchand, B., et al. Identification and Biochemical Characterization of a New Class of HIV Inhibitors: Nucleotide-competing Reverse Transcriptase Inhibitors. 12^{th} Conference on Retroviruses and Opportunistic Infections: Feb. 22-25, 2005; Boston, Mass., USA. Oral presentation, abstract 156.
2 Ehteshami, M., Deval, J., Barry, S., et al. Nucleotide-Competing Reverse Transcriptase Inhibitors Form a Stable Dead-End-Complex with the HIV-1 Enzyme. 13^{th} Conference on Retroviruses and Opportunistic Infections: Feb. 5-8, 2006; Denver, Colo., USA. Oral presentation, abstract F-109.
3 Hertogs, K., de Béthune, M.-P., Miller, V., et al. A Rapid Method for Simultaneous Detection of Phenotypic Resistance to Inhibitors of Protease and Reverse Transcriptase in Recombinant Human Immunodeficiency Virus Type 1 Isolates from Patients Treated with Antiretroviral Drugs. Antimicrob Agents Chemother 1998: 42; 269-276.
4 Stone, C., Ait-Khaled, M., Craig, C., et al. Human Immunodeficiency Virus Type 1 Reverse Transcriptase Mutation Selection during In Vitro Exposure to Tenofovir Alone or Combined with Abacavir or Lamivudine. Antimicrob Agents Chemother 2004: 48; 1413-1415.

Lloyd, R., Huong, J., Rouse, E., et al. HIV-1 RT Mutations K70E and K65R are Not Present on the Same Viral Genome when Both Mutations are Detected in Plasma. 45^{th} Interscience Conference on Antimicrobial Agents and Chemotherapy (ICAAC): Dec. 16-19, 2005; Washington, D.C., USA. Poster H-1066.

1. The use of an NcRTI for the manufacture of a medicament for treating patients infected with HIV that has a K65R mutation in the viral genome encoding for reverse transcriptase.

2. The use of a combination of HIV inhibitors, at least one of which is an NcRTI, for the manufacture of a medicament for treating patients infected with HIV that has a K65R mutation in the viral genome encoding for reverse transcriptase.

3. The use according to claims 1 or 2 wherein the NcRTI is a compound of formula (I) 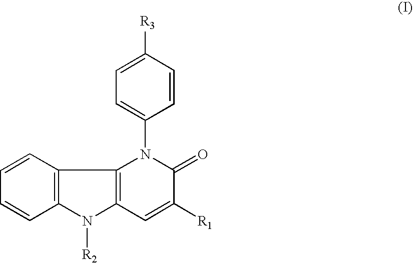
wherein
R₁ is cyano, methyloxycarbonyl, methylaminocarbonyl, ethyloxycarbonyl and ethylaminocarbonyl, more in particular wherein R₁ is cyano, ethyloxycarbonyl and ethylaminocarbonyl, even more in particular wherein R₁ is cyano.
R₂ is hydrogen, C₁₋₆alkyl optionally substituted with cyano, NR₄ₐR_{4b}, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxycarbonyl; in particular R₂ is C₁₋₆alkyl, hydrogen, C₂₋₆alkenyl;
R₃ is nitro, cyano, amino, halo, hydroxy, C₁₋₄alkyloxy, hydroxycarbonyl, aminocarbonyl, C₁₋₄alkyloxycarbonyl, mono- or di(C₁₋₄alkyl)aminocarbonyl, C₁₋₄alkylcarbonyl, or Het₁; in particular R₃ is nitro;
R₄ₐ and R_{4b} independently from each other are hydrogen, C₁₋₄alkyl or C₁₋₄alkyl substituted with amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl; in particular R₄ₐ and R_{4b} independently from each other are hydrogen, C₁₋₄alkyl;
Het₁ is furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, each optionally substituted with C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyano-C₁₋₄alkyl, mono- or di(C₁₋₄alkyl)amino, aminoC₁₋₄alkyl, mono- or di(C₁₋₄alkyl)-aminoC₁₋₄alkyl, arylC₁₋₄alkyl, aminoC₂₋₆alkenyl, mono- or di(C₁₋₄alkyl)amino-C₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)-aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl; in particular Het₁ is furanyl, thienyl, pyrrolyl, oxazolyl, thiazolyl, imidazolyl, isoxazolyl, isothiazolyl, pyrazolyl, oxadiazolyl, thiadiazolyl, triazolyl, each optionally substituted with C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, as well as the pharmaceutically acceptable addition salts thereof.

4. The use according to claim 3 wherein R² is hydrogen, C₁₋₆alkyl optionally substituted with NR₄ₐR_{4b}, pyrrolidinyl, piperidinyl, homopiperidinyl, piperazinyl, 4-(C₁₋₄alkyl)-piperazinyl, morpholinyl, aryl, furanyl.

5. The use according to claims 1 or 2 wherein the NcRTI is 5-methyl-1-(4-nitro-phenyl)-2-oxo-2,5-dihydro-1H-pyrido[3,2-b]indole-3-carbonitrile.

6. A method for treating patients infected with HIV that has a K65R mutation in the viral genome encoding for reverse transcriptase, said method comprising administering an effective amount to said patients of an NcRTI.

7. A method for treating patients infected with HIV that has a K65R mutation in the viral genome encoding for reverse transcriptase, said method comprising administering an effective amount to said patients of a combination of HIV inhibitors, at least one of which is an NcRTI.

8. A method according to claims 5 or 6 wherein the NcRTI is 5-methyl-1-(4-nitro-phenyl)-2-oxo-2,5-dihydro-1H-pyrido[3,2-b]indole-3-carbonitrile.

9. A method for treating patients infected with HIV mutants that has developed resistance towards NcRTI and in particular has a M184V and/or Y115F mutation in the viral genome encoding for reverse transcriptase, said method comprising administering an effective amount to said patients of tenofovir or of a teneofovir derivative, in particular tenofovir disoproxil fumarate (TDF).
